(19) United States
(12) Patent Application Publication
(10) Pub. No.: US 2020/0371115 A1
(43) Pub. Date: Nov. 26, 2020

(54) APOE MODIFICATIONS AND USES THEREOF

(71) Applicant: GEORGETOWN UNIVERSITY, Washington, DC (US)

(72) Inventors: George William Rebeck, Washington, DC (US); Sarah Flowers, Washington, DC (US)

(73) Assignee: GEORGETOWN UNIVERSITY, Washington, DC (US)

(21) Appl. No.: 16/638,962

(22) PCT Filed: Aug. 15, 2018
(86) PCT No.: PCT/IB2018/056138

§ 371 Date: Feb. 13, 2020

Related U.S. Application Data
(60) Provisional application No. 62/545,839, filed on Aug. 15, 2017.

(51) Int. Cl. G01N 33/68

(52) U.S. Cl. CPC G01N 33/6896; G01N 2333/775; G01N 2800/2821; G01N 2440/38; G01N 2400/02

(57) ABSTRACT

Provided herein are methods for detecting a cerebrospinal fluid-specific (CSF-specific) glycoform of Apolipoprotein E (APOE) in a subject.

This application claims the benefit of U.S. Provisional Application No. 62/545,839, filed Aug. 15, 2017 which is hereby incorporated herein in its entirety by this reference.

### BACKGROUND

Alzheimer's Disease is the sixth leading cause of death in the United States. Alzheimer's Disease is a progressive type of dementia that causes problems with memory, thinking and behavior that gradually worsen over a number of years. Those with Alzheimer's Disease live an average of eight years after their symptoms become noticeable to others.

### SUMMARY

Provided herein are methods of detecting a cerebrospinal fluid-specific (CSF-specific) glycoform of Apolipoprotein E (APOE) in a sample from a subject. The sample can be, for example, a CSF sample or a plasma sample. The methods optionally comprise obtaining a plasma sample from the subject and detecting a CSF-specific glycoform of APOE in the plasma sample.

Also provided are methods of diagnosing Alzheimer's disease or a risk of Alzheimer's disease in a subject. The methods comprise obtaining a plasma sample from a subject, detecting a CSF-specific glycoform form of APOE in the plasma sample, and diagnosing the subject as having Alzheimer's disease or at risk of developing Alzheimer's disease when a difference in the level of the CSF-specific form of APOE, and/or the glycosylation pattern of the CSF-specific form of APOE as compared to a control level of the CSF-specific form of APOE, and/or a control glycosylation pattern of the CSF-specific form of APOE is detected.

Further provided are methods of determining the progression of Alzheimer's disease or an increase in the risk of developing Alzheimer's disease in a subject. The methods comprise obtaining a first plasma sample from the subject; detecting a level and/or a glycosylation pattern of a CSF-specific glycoform of APOE in the plasma sample; obtaining a second plasma sample from the subject; d) detecting a second level and/or second glycosylation pattern of a CSF-specific form of APOE in the plasma sample; comparing the first level and/or glycosylation pattern of the CSF-specific glycoform of APOE with the second level and/or glycosylation pattern of the CSF-specific glycoform of APOE, wherein if the level and/or glycosylation pattern of the CSF-specific glycoform of APOE in the second biological sample is more similar to control indicating the progression of Alzheimer's disease or increased risk of Alzheimer's disease, as compared to the first biological sample, Alzheimer's disease has progressed or the risk of developing Alzheimer's disease has increased in the subject.

### DESCRIPTION OF THE FIGURES

FIGS. 1A-B show trypsinization of N-terminal pep 1-15, and a comparison of peptide 1-15 and 2-15. Extracted ion chromatograms (XIC) of peptide 1-15 and peptide 2-15 from (Figure A) CSF and (Figure B) plasma. The N-terminal peptide, KVEQAVETEPEPELR (SEQ ID NO: 1), is a miscleavage. The cleavage of the N-terminal lysine (K) is highly inefficient even given the excess trypsin environment used in these experiments due to the high lysine content and difficult cleavage of APOE. Peptide 1-15 is orders of magnitude higher in quantity compared to peptide 2-15 in both the CSF and plasma samples.

FIGS. 2A-D show glycosylation of the N-Terminal 1-15 peptide. FIG. 2A shows MS/MS spectra of unglycosylated VEQAVETEPEPELR (SEQ ID NO: 2) peptide. FIG. 2B shows MS/MS spectra of peptide with NeuAcα2-3Galβ1-3GalNAcα1- attached showing peaks of NeuAc (m/z 274.09 and 292.10) as well as Galβ1-3GalNAc (m/z 366.14) and GalNac (m/z 204.09). FIG. 2C shows XICs of unglycosylated and sialylated core 1 glycosylated 1-15 peptide from CSF (n=2) and FIG. 2D shows XICs from plasma (n=2). CSF shows a higher proportion of unglycosylated peptide with the glycosylated peptide. The background is low except for the unglycosylated peptide in plasma which shows higher background generally as well as an addition unrelated peak (confirmed to be unrelated by MS/MS) at 15.3 min. VEQAVETEPEPELR (SEQ ID NO: 2) peptide and VEQAVETEPEPELR (SEQ ID NO: 2) peptide with NeuAcα2-3Galβ1-3GalNAcα1-structure attached are shown. All masses are observed masses.

FIGS. 3A-B show MS/MS of NeuAcα2-3Galβ1-3GalNAcα1- and glycosylated and KVEQAVETEPEPELR (SEQ ID NO: 1) peptide 1-15. Standard plasma APOE (rPeptide) was used in a directed approach to acquire MS/MS of the glycosylated peptide 1-15 to confirm glycan structure. FIG. 3A confirms the linear sialylated core 1 structure on peptide 1-15. The fragment at m/z 454.15 confirms the linear NeuAcα2-3Galβ1-3GalNAcα1-structure. FIG. 3B confirms the site can hold the disialylated structure although it is of very low abundance and can only be detected in a standard APOE from plasma at higher concentrations with a directed method and not from the patient samples.

FIGS. 4A-C show a comparison of the glycoprofiles of CSF and plasma APOE. FIG. 4A is a schematic of APOE domain structures showing amino acids 112 and 158, the receptor binding domain, the hinge, and the lipid binding domain. The schematic below shows glycosylated tryptic peptides in grey with position of glycosites. FIG. 4B shows the average (n=2) of the percentage of identified peptide that was unglycosylated or glycosylated. FIG. 4C shows ISOGlyP results for APOE glycosites. T refers to the GalNAc-T. Results are shown as EVP (enhancement value product) which refers to the preference a GalNAc-T shows toward glycosylating a glycosite, greater than one correlates with a positive likelihood of that glycosite being able to be glycosylated by that GalNAc-T and less than 1 suggests a negative correlation with that specific GalNAc-T. The EVP does not correlate with the probability of that glycosite being glycosylated, it is GalNAc-T specific as it only considers ten of the twenty known enzymes, others may contribute to glycosylation at any particular glycosite.

FIGS. 5A-F show structures of CSF and plasma glycovariants. All APOE structures are APOE3 NMR crystal structure 2L7B with the following glycosylation modelled at the indicated glycosites. FIG. 5A shows Thr8 glycosylated with NeuAcα2-3Galβ1-3GalNAcα1-, most abundant in plasma APOE. FIG. 5B shows Thr194 glycosylated with NeuAcα2-3Galβ1-3GalNAcα1-, identified in plasma and CSF APOE. C-terminal glycosites most abundant in CSF APO are shown in FIG. 5C (Thr289 glycosylated with NeuAcα2-3Galβ1-3GalNAcα1-); FIG. 5D (Ser296 glycosylated with NeuAcα2-3Galβ1-3GalNAcα1-); FIG. 5E (Ser290 glycosylated with NeuAcα2-3Galβ1-3GalNAcα1-); and FIG. 5F (Ser290 glycosylated with NeuAcα2-3Galβ1-3(NeuAcα2-6)GalNAcα1-). The protein backbone is displayed in NewCartoon. The N-terminal region with the LDL receptor binding domain are shown, as are the hinge region and the C-terminal with the lipid binding region. Amino acid 112 and amino acid 158 are also shown. The glycan is displayed in CPK.

FIGS. 6A-C show glycosylation of N-terminal QQTEWQSGQR (SEQ ID NO: 3) peptide 16-25 in an APOE standard. This glycosylated peptide was identified in standard APOE isolated from plasma when a higher quantity of sample was analysed but was not identified in the patient samples. The peptide includes two possible glycosylation sites, Thr18 has previously been suggested as a glycosylation site on APOE. FIG. 6A shows MS/MS spectra of unglycosylated pep16-25. FIG. 6B shows a MS/MS spectra of the peptide with sialylated core 1 structure attached showing peaks of NeuAc (m/z 274.09 and 292.10) as well as Galβ1-3GalNAc (m/z 366.14) and GalNac (m/z 204.09). FIG. 6C shows MS/MS spectra of pep16-25 with NeuAcα2-3Galβ1-3(NeuAcα2-6)GalNAcα1- attached showing peaks of NeuAc (m/z 274.09 and 292.10) as well as Galβ1-3GalNAc (m/z 366.14), NeuAcα2-6GalNAc (m/z 495.18), and GalNac (m/z 204.09).

FIGS. 7A-E show glycosylation of the hinge domain 192-206 peptide. FIG. 7A shows MS/MS spectra of core 1 glycosylated AATVGSLAGQPLQER pep192-206 (SEQ ID NO: 4) showing peaks Galβ1-3GalNAc (m/z 366.14) and GalNac (m/z 204.09). FIG. 7B shows MS/MS spectra of the peptide with NeuAcα2-3Galβ1-3GalNAcα1- attached showing peaks of NeuAc (m/z 274.09 and 292.10) as well as Galβ1-3GalNAc (m/z 366.14) and GalNac (m/z 204.09). Linear structure is confirmed by the m/z 454.15 NeuAcα2-3Gal fragment. FIG. 7C shows MS/MS spectra of peptide with NeuAcα2-3Galβ1-3(NeuAcα2-6)GalNAcα1- attached showing peaks of NeuAc (m/z 274.09 and 292.10) as well as Galβ1-3GalNAc (m/z 366.14), NeuAcα2-6GalNAc (m/z 495.18), and GalNac (m/z 204.09). XICs of unglycosylated and glycosylated 192-206 peptide from CSF (n=2) are shown in FIG. 7D and from plasma (n=2) are shown in FIG. 7E. AATVGSLAGQPLQER peptide (SEQ ID NO: 4) is shown. A peptide with core 1 structure attached is shown in the far left of FIGS. 7D and 7E. The peptide with sialylated core 1 structure attached is shown as is the peptide with disialylated core 1 structure attached. All masses are observed masses.

FIGS. 8A-D show MS/MS of glycosylation of C-terminal VQAAVGTSAAPVPSDNH peptide 283-299 (SEQ ID NO: 5) from CSF APOE. FIG. 8A shows MS/MS spectra of unglycosylated VQAAVGTSAAPVPSDNH peptide (SEQ ID NO: 5). FIG. 8B shows MS/MS spectra of core 1 glycosylated pep283-299 showing peaks Galβ1-3GalNAc (m/z 366.14) and GalNac (m/z 204.09). FIG. 8C shows MS/MS spectra of the peptide with NeuAcα2-3Galβ1-3GalNAcα1- attached showing peaks of NeuAc (m/z 274.09 and 292.10) as well as Galβ1-3GalNAc (m/z 366.14) and GalNac (m/z 204.09). Linear structure is confirmed by the m/z 454.15 NeuAcα2-3Gal fragment. FIG. 8D shows MS/MS spectra of pep283-299 with NeuAcα2-3Galβ1-3(NeuAcα2-6)GalNAcα1- attached showing peaks of NeuAc (m/z 274.09 and 292.10) as well as Galβ1-3GalNAc (m/z 366.14), NeuAcα2-6GalNAc (m/z 495.18), and GalNac (m/z 204.09).

FIGS. 9A-B show glycosylation of the lipid binding domain 283-299 peptide in CSF. FIG. 9A shows XIC of NeuAcα2-3Galβ1-3GalNAcα1- glycosylated 283-299 peptide from CSF (n=2). FIG. 9B shows XIC of NeuAcα2-3Galβ1-3(NeuAcα2-6)GalNAcα1-glycosylated 283-299 peptide from CSF (n=2). Each chromatogram shows three glycoforms of the 283-299 peptide for the two glycan structures as confirmed by MS/MS. A peptide with sialylated core 1 structure attached and a peptide with disialylated core 1 structure attached are shown.

FIGS. 10A-B show MS/MS of glycosylated C-terminal VQAAVGTSAAPVPSDNH peptide 283-299 (SEQ ID NO: 5) showing loss of glycosylation peaks. The loss of attached glycan is observed by the associated b-ion with the loss of 18 Da. These MS/MS are from the second and third peaks of from NeuAcα2-3Galβ1-3GalNAcα1- glycosylated pep283-299 CSF as shown in FIG. 3a of the article. FIG. 10A shows MS/MS of the NeuAcα2-3Galβ1-3GalNAcα1- glycosylated pep283-299, which is from the second peak (RT). It shows the low abundance b7-18 m/z 609.3355 (expected, observed m/z are shown in figure) peak indicating that Thr289 of this peptide was glycosylated. FIG. 10B shows MS/MS of the glycosylated pep283-299, which is from the third peak. It shows the b8-18 m/z 696.3675 indicating that the Ser290 of this peptide was glycosylated. As these two spectra indicate the site Thr289 and Ser290 sites peak indicating the NeuAcα2-3Galβ1-3GalNAcα1- linear isomer, the first peak of lower intensity, which did not show associated loss of glycosylation for any site can, by exclusion, be define as glycosylated at Ser296. It should be noted that the branched form of the monosialylated core one structure, not confirmed here to be attached to APOE at detectable levels, could also account for an isomeric form of the peptide.

FIG. 11 shows structural changes due to sialylated glycovariants in the lipid binding domain. The APOE3 crystal structure with a disialylated core 1 glycan (Neu5Acα2-3Galβ1-4[Neu5Acα2-6]GalNAcα) modelled at Ser290 (displayed as licorice with 3D-SNFG icons), which is part of the C-terminal lipid binding region (purple). Relative to the monosialylated core 1 structure, the α2-6 linked Neu5Ac could potentially form interactions with the C-terminal domain via V232 and D230, as well as the LDL receptor binding via E132 and E131.

### DESCRIPTION

Provided herein is a method of detecting a cerebrospinal fluid-specific (CSF-specific) glycoform of Apolipoprotein E (APOE) in a sample (e.g., plasma or CSF) from a subject comprising obtaining a plasma sample from the subject and detecting a CSF-specific glycoform of APOE in the plasma sample.

As used throughout, a glycoform of APOE is an isoform of APOE that differs with respect to the number or type of glycans attached to APOE. In the methods provided herein, one or more CSF-specific glycoforms can be detected in a sample from a subject. In some methods, a CSF-specific glycoform of APOE is detected in a CSF sample from the subject instead of or in addition to detection of a CSF-specific glycoform of APOE in a plasma sample from the subject.

Any of the methods provided herein, can further comprise determining the level of the CSF-specific glycoform of APOE and/or the glycosylation pattern of the CSF-specific glycoform of APOE in the plasma sample. In some methods, the CSF-specific glycoform of APOE is an APOE glycoform that differs in glycosylation as compared to a control plasma-specific glycoform of APOE. In some methods, the CSF-specific glycoform of APOE is an APOE glycoform that differs in glycosylation as compared to a control CSF-specific glycoform of APOE. In other examples, the CSF-specific glycoform of APOE detected in the subject differs in glycosylation as compared to a control plasma-specific glycoform of APOE and differs in glycosylation as compared to a control CSF-specific glycoform of APOE. In any of the methods described herein, a difference in the level of the CSF-specific form of APOE can be an increase or a decrease in the level of the CSF-specific form of APOE.

In any of the methods provided herein, the difference in the glycosylation pattern of the CSF-specific glycoform of APOE can be a difference in the number of glycosylated O-linked glycosylation sites, a difference in the type of O-glycan at one or more glycosylation sites, a difference in the amount of glycosylation at one or more O-linked glycosylation sites and/or a difference in sialylation at one or more O-linked glycosylation sites. It is understood that a difference in the number of glycosylated O-linked glycosylation sites, a difference in the type of O-glycan at one or more glycosylation sites, a difference in the amount of glycosylation at one or more O-linked glycosylation sites and/or a difference in sialylation at one or more O-linked glycosylation sites can occur along with no change or difference in one or more of the number of glycosylated O-linked glycosylation sites, the type of O-glycan at one or more glycosylation sites, the amount of glycosylation at one or more O-linked glycosylation sites and/or a difference in sialylation at one or more O-linked glycosylation sites.

As used throughout, O-linked glycans are all based on a core structure with N-acetylgalactosamine (GalNAc) units in 0-linkage with serine or threonine. Briefly, biosynthesis begins with the addition of an N-acetyl galactosamine (GalNac) to the hydroxyl group of a serine or threonine by one of twenty redundant UDP-GalNac:polypeptide N-acetylglucosaminyl-transferases (GalNac-T). The core 1 structure (Galβ1-3GalNAcα1-) is then completed by the addition of a galactose by core 1 β3-galactosyltransferase. Sialic acid can be added by a range of linkage and monosaccharide dependent sialytransferases, commonly creating 2-3 or 2-6 linkages with the adjoining monosaccharide generating, for example, and not to be limiting, the linear sialylated (NeuAcα2-3Galβ1-3GalNAcα1-) or disialylated (NeuAcα2-3Galβ1-3(NeuAcα2-6)GalNAcα1-) core 1 structures. In the methods provided herein, a difference in sialylation can be a difference in the total amount of sialylation, a difference in a (2,3)-linked sialylation and/or a difference in a (2,6)-linked sialylation. As used throughout, a (2,3)-linked sialylation is the addition of sialic acid to galactose (Gal). Also, as used throughout, a (2,6)-linked sialylation is the addition of sialic acid to N-acetylglucosamine (GalNac).

In some methods the difference in glycosylation pattern is a difference in the amount of glycosylation, type of O-glycan, and/or amount of sialyation at Thr8, Thr18, Thr194, Ser197, Thr289, Ser290 and/or Ser296 of the CSF-specific APOE glycoform. In some methods the difference in glycosylation pattern is a difference in the amount of glycosylation, type of O-glycan, and/or amount of sialyation at Thr289, Ser290 and/or Ser296 of the CSF-specific APOE glycoform.

In some methods, the difference in glycosylation pattern is a difference in the amount of glycosylation, type of O-glycan, and/or amount of sialyation in the C-terminus of the CSF-specific glycoform of APOE as compared to a control CSF-specific glycoform of APOE or a control plasma-specific glycoform of APOE.

The methods provided herein include methods where the CSF-specific glycoform of APOE is detected and one or more plasma-specific forms of APOE are not detected. Any of the methods provided herein can further comprise detecting one or more plasma-specific glycoforms of APOE.

In the methods provided herein, the CSF-specific glycoform of APOE and/or the plasma-specific glycoform of APOE can be detected using an assay selected from the group consisting of Western blot, enzyme-linked immunosorbent assay (ELISA), enzyme immunoassay (EIA), radioimmunoassay (MA). The CSF-specific glycoform of APOE and/or the plasma-specific glycoform of APOE can also be detected by mass spectroscopy.

As used throughout, by subject is meant an individual. Preferably, the subject is a mammal such as a primate, and, more preferably, a human. Non-human primates are subjects as well. The term subject includes domesticated animals (such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

In any of the methods set forth herein, the subject can be a subject that has at least one copy of the APOE4 allele or a subject that lacks copies of the APOE4 allele. The subject can also be a subject that has two copies of the APOE4 allele, a subject that has one copy of the APOE4 allele and one copy of the APOE3 allele, a subject that has one copy of the APOE4 allele and one copy of the APOE2 allele, a subject that has two copies of the APOE3 allele or a subject that has two copies of the APOE3 allele. Compared to the most common APOE3 allele, each APOE4 allele increases risk of AD 2.5 times, while the APOE2 allele decreases risk. The APOE alleles encode a mature secreted protein of 299 amino acids, with single amino acid substitutions accounting for each isoform (E2: Cys112, Cys158; E3: Cys112; Arg158; E4: Arg112, Arg158) See Rall et al., Human apolipoprotein E. the complete amino acid sequence, J. Biol. Chem. 257: 4171-4178 (1982), incorporated herein in its entirety by this reference. Thus, one or more of APOE2-4 can be assessed according to the methods described herein. The subject can also be a subject diagnosed with Alzheimer's disease or at risk for Alzheimer's disease. Identification of a subject at risk for Alzheimer's disease can be determined based, for example, on family history, genetic predisposition, or history of brain injuries (e.g., concussions).

Further provided is a method of detecting the ratio of glycosylated APOE to non-glycosylated APOE in the subject comprising obtaining a sample from the subject, detecting the total amount of APOE, glycosylated APOE and non-glycosylated APOE and determining the ratio of glycosylated APOE to non-glycosylated APOE in the sample. Similarly the ratio of glycosylated APOE to total APOE can be determined or the ratio of glycosylated CSF-specific APOE to total glycosylated APOE can be determined.

### Methods for Diagnosing and Treating Alzheimer's Disease

Also provided is a method of diagnosing Alzheimer's disease or a risk of Alzheimer's disease in a subject comprising obtaining a plasma sample from a subject; detecting a CSF-specific glycoform form of APOE in the plasma sample; diagnosing the subject as having Alzheimer's disease or at risk of developing Alzheimer's disease when a difference in the level of the CSF-specific form of APOE, and/or the glycosylation pattern of the CSF-specific form of APOE as compared to a control level of the CSF-specific form of APOE, and/or a control glycosylation pattern of the CSF-specific form of APOE is detected.

The control level of the CSF-specific form of APOE can be a level of one or more CSF-specific forms of APOE from a subject or a level that corresponds to a subject or a population of subjects that does not have Alzheimer's disease or a risk of Alzheimer's disease that is greater than the risk of Alzheimer's disease in the general population.

The methods of diagnosing Alzheimer's or a risk of Alzheimer's disease in a subject can further comprise administering one or more agents that slow the progression or delays the development of Alzheimer's disease. These agents include, but are not limited to, one or more agents selected from the group consisting of a nonsteroidal anti-inflammatory drug (MAID), a tyrosine kinase inhibitor, an acetylcholinesterase inhibitor, and an NMDA receptor inhibitor. Examples of NSAIDs include, but are not limited to, aspirin, celecoxib, diclofenac, diflunisal, etodolac, ibuprofen and indomethacin. Examples, of tyrosine inhibitors include, but are not limited to, nilotinib, bosutinib and imatinib and derivatives thereof. Examples of acetylcholinesterase inhibitors include, but are not limited to, donepezil, tacrine, rivastigimine and metrifonate and derivatives thereof. Examples of NMDA receptor inhibitors include, but are not limited to, memantine and derivatives thereof.

Further provided is a method of determining the progression of Alzheimer's disease or an increase in the risk of developing Alzheimer's disease in a subject comprising obtaining a first plasma sample from the subject; detecting a level and/or a glycosylation pattern of a CSF-specific glycoform of APOE in the plasma sample; obtaining a second plasma sample from the subject; detecting a second level and/or second glycosylation pattern of a CSF-specific form of APOE in the plasma sample; comparing the first level and/or glycosylation pattern of the CSF-specific glycoform of APOE with the second level and/or glycosylation pattern of the CSF-specific glycoform of APOE, wherein if the level and/or glycosylation pattern of the CSF-specific glycoform of APOE in the second biological sample is more similar to control indicating the progression of Alzheimer's disease or increased risk of Alzheimer's disease, as compared to the first biological sample, Alzheimer's disease has progressed or the risk of developing Alzheimer's disease has increased in the subject.

A control level or glycosylation pattern indicating the progression of Alzheimer's disease or increased risk of Alzheimer's disease, as compared to the first biological sample can be, for example, a control level or glycosylation pattern from a subject that has a particular stage of Alzheimer's or a level or glycosylation pattern corresponding to a subject or a population of subjects with a particular stage of Alzheimer's disease. The control can also be from a subject at increased risk of Alzheimer's disease, as compared to the general population, or a level or glycosylation pattern corresponding to a subject or a population of subjects at increased risk for Alzheimer's disease.

In methods for determining the progression of Alzheimer's disease, levels and/or glycosylation patterns CSF-specific form of APOE in the subject can be compared with levels and/or glycosylation patterns of CSF-specific form of APOE from a subject that has at least one copy of an APOE2 allele, a subject that has at least one copy of an APOE3 allele, and/or a subject that has at least one copy of an APOE4 allele.

In the methods for determining progression of Alzheimer's disease, the first and second sample can be taken, days, weeks months, or years apart. Samples can be taken from the subject throughout their life, for example, from about 40 years of age to about 100 years of age to determine if the disease has progressed or an increase in the risk of developing of Alzheimer's disease has occurred. Therefore, two, three, four, five, six, seven, eight, nine, ten samples or greater, taken from the subject at intervals, can be analyzed to detect differences between the two most recent samples as well as other samples previously obtained from the subject.

Also provided is a method for determining the efficacy of a selected treatment for slowing the progression or delaying the development of Alzheimer's disease in a subject comprising obtaining a first plasma sample from the subject before the selected treatment; detecting a level and/or a glycosylation pattern of a CSF-specific glycoform in the first sample; treating the subject with the selected treatment; obtaining a second plasma sample from the subject after the selected treatment; detecting a level and/or glycosylation pattern of a CSF-specific glycoform of APOE in the second sample; comparing the level and/or glycosylation pattern of the CSF-specific glycoform detected in the first and second samples to determine whether the level and/or glycosylation pattern is the same or whether the level and/or glycosylation pattern detected in the second samples is more similar to control, a level and/or glycosylation pattern more similar to control indicating that the selected treatment is effective for treating or preventing Alzheimer's disease.

In the methods of determining the efficacy of a selected treatment, the control can be a level or glycosylation pattern from a subject that does not have Alzheimer's disease, a control level or glycosylation pattern from a subject that has been successfully treated for Alzheimer's disease, or a control level or glycosylation pattern from a subject, wherein the progression of Alzheimer's disease has been delayed by the selected treatment.

Any of the methods described herein can be combined with other tests to diagnose or determine the progression of Alzheimer's disease. For example, and not to be limiting, methods of diagnosing, determining the progression of Alzheimer's disease, or determining the effectiveness of a selected treatment can further comprise blood tests, brain imaging, mental status testing, mood testing, a physical exam and/or a neurological exam.

The agents described herein can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22nd edition, Loyd V. Allen et al, editors, Pharmaceutical Press (2012)

Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Administration can be carried out using therapeutically effective amounts of the agents described herein for periods of time effective to treat Alzheimer's disease or delay the progression of Alzheimer's disease. The effective amount can be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent can be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any appropriate route of administration can be employed, for example, parenteral, intravenous, subcutaneous, intramuscular, intraventricular, intracorporeal, intraperitoneal, rectal, or oral administration. Administration can be systemic or local. Pharmaceutical compositions can be delivered locally to the area in need of treatment, for example by topical application or local injection. Multiple administrations and/or dosages can also be used. Effective doses for any of the administration methods described herein can be extrapolated from dose-response curves derived from in vitro or animal model test systems.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications can be made. Accordingly, other embodiments are within the scope of the following claims.

### Examples

Apolipoprotein E (APOE) associates with an array of lipoproteins. Cerebrospinal fluid (CSF) APOE binds only with high-density lipoproteins (HDL), while plasma APOE attaches to widely sized lipoproteins. APOE O-glycosylation was analyzed by detailed mass spectrometry. Plasma APOE held more abundant, sialylated core 1 (NeuAcα2-3Galβ1-3GalNAcα1-)N-terminal glycosylation (Thr8), while CSF APOE held more abundant C-terminal glycosylation (Thr289, Ser290 and Ser296), with sialylated and disialylated (NeuAcα2-3Galβ1-3(NeuAcα2-6)GalNAcα1-) core 1 structures. APOE in both CSF and plasma were hinge domain glycosylated (Thr194). Compared to plasma, CSF APOE had a much higher proportion of sialylated glycan structures—previously shown to improve the binding of HDL—within the lipid binding domain loop C. The structural effects of glycosylation were modeled using GLYCAM-Web glycoprotein builder. These data define novel, specific APOE glycoforms in human CSF and plasma, allowing monitoring for brain APOE isoforms that strongly affect risk of late-onset Alzheimer's disease.

Apolipoprotein E (APOE) is the most significant genetic risk factor for late onset Alzheimer's disease (AD), the most common form of AD. Compared to the most common APOE3 allele, each APOE4 allele increases risk of AD 2.5 times, while the APOE2 allele decreases risk (Wu et al., ApoE2 and Alzheimer's disease: time to take a closer look, Neural Regeneration Research 11, 412-413, doi:10.4103/1673-5374.179044 (2016)). The APOE alleles encode a mature secreted protein of 299 amino acids, with single amino acid substitutions accounting for each isoform (E2: Cys112, Cys158; E3: Cys112; Arg158; E4: Arg112, Arg158) (Rall et al., Human apolipoprotein E. The complete amino acid sequence, J Biol Chem 257, 4171-4178 (1982)).

APOE is essential for lipid transport in the brain and plasma and is able to bind lipoproteins of diverse size and shape to carry out its complex roles. In the brain, APOE is expressed by astrocytes, microglia and the choroid plexus. APOE associates with small high density lipoproteins (HDL) in the brain, which increase in size in the cerebrospinal fluid (CSF). CSF APOE diffuses into the plasma via arachnoid granulations in the sagittal sinus. The APOE lifecycle is more complex in the periphery taking part in the HDL, exogenous and endogenous cholesterol pathways. Expressed primarily by hepatocytes as well as macrophages, APOE is found on plasma HDL. The endogenous pathway begins with nascent very low density lipoprotein (VLDL) production in the liver containing APOB100, APOCI, APOCII and APOE and released into the plasma. Plasma VLDL are hydrolysed to intermediate density lipoproteins, which can contain multiple APOE molecules before APOE is lost. The exogenous pathway involves intestinal derived chylomicrons containing APOB48, which enter the plasma where they gain APOE from circulated HDL and reduce in size by lipoprotein lipase until remnant particles are removed by the liver via APOE receptor binding. APOE is an O-glycoprotein that contains an N-terminal four helix receptor-binding domain, a central flexible hinge region and a C-terminal triple helix lipid-binding domain. On binding to lipoprotein, APOE undergoes a conformational change involving the hinge region. This alteration exposes the previously buried receptor binding domain, ensuring that optimal binding to members of the (low density lipoprotein) LDL receptor family is achieved only with fully lipidated APOE. Given the flexibility of the APOE structure and the significance of this characteristic to its function, the position and nature of its O-glycosylation is particularly important.

Mucin-like O-glycosylation is made up of 8 core structures, with core 1-4 principally in humans, that can then be extended by the addition of other monosaccharides, such as sialic acid (N-Acetylneuraminic acid), often biologically significant due to its negative charge. Biosynthesis begins with the addition of an N-acetyl galactosamine (GalNAc) to the hydroxyl group of a serine or threonine by one of twenty redundant UDP-GalNAC:polypeptide N-acetylgalactosaminyl-transferases (GalNAc-T). These enzymes differ in tissue expression and substrate specificities; for example, GalNAcT1 and 2 are expressed ubiquitously, whereas others show narrow tissue particularity. The core 1 structure (Galβ1-3GalNAcα1-), a simple and very common structure, is then completed by the addition of a galactose by core 1 β3-galactosyltransferase. Sialic acid can be added by a range of linkage and monosaccharide dependent sialytransferases commonly creating 2-3 or 2-6 linkages with the adjoining monosaccharide generating, for example, the linear sialylated (NeuAcα2-3Galβ1-3GalNAcα1-) or disialylated (NeuAcα2-3Galβ1-3(NeuAcα2-6)GalNAcα1-) core 1 structures.

Early work first identified that Thr194, within the hinge region, could be glycosylated in plasma APOE (Wernette-Hammond et al., Glycosylation of human apolipoprotein E. The carbohydrate attachment site is threonine 194, J Biol Chem 264, 9094-9101 (1989)). While there is evidence that intracellular APOE is more heavily glycosylated than secreted or plasma APOE, there has been no comprehensive comparative study to characterize the glycans attached and site-occupancy at all sites between the CSF and plasma. To better understand APOE glycosylation and its potential role on its varied functions, glycoproteomic analyses of APOE isolated from CSF and plasma APOE from normal individuals was performed. These glycoprofiles were used to define and model CSF and plasma specific glyco-APOE variants, including how specific glycans affect the protein structure.

### Sample Information

Human lumbar puncture CSF samples (n=2) were from Washington University in St Louis, collected as control samples as part of a study on CSF biomarkers in Alzheimer's disease. All samples and clinical information were anonymized, all individuals gave written informed consent and the study was approved by the Human Research Protection Office at Washington University. Individuals were confirmed to be normal by negative amyloid Pittsburgh compound B (PiB) positron emission tomography (PET). Human plasma samples (n=2) were collected in EDTA. Samples were from the Georgetown Brain Bank tissue and biofluid repository. All samples and clinical information were anonymized, all individuals gave written informed consent and the study was approved by the Institutional Review Board at Georgetown University Medical Center. Plasma normal controls were confirmed to be normal by MMSE. Standard APOE was from fresh human plasma (rPeptide, Watkinsville, Ga.).

### Immunoprecipitation

Samples were precleared with Protein A-Sepharose® beads (Sigma, St. Louis, Mo.) for 1 h at 4° C. with rotation. An excess of beads was used to preclear the higher immunoglobulin content of plasma. Protease (Pierce Mini Tablets with EDTA, 88665, Dallas, Tex.) and phosphatase (Pierce phosphatase inhibitor mini tablets 88667, Dallas, Tex.) inhibitors were added to the IP buffer (50 mM Tris-HCl pH 8, 150 mM NaCl, 1% NP40). The amount of antibody used for IP was optimized for each sample type. Precleared samples were incubated with fresh beads and goat polyclonal anti-APOE (K74190G, Meridian Life Science, Memphis, Tenn.) in IP buffer for 16 h at 4° C. with rotation. Beads were washed five times with IP buffer and sample removed from beads with NuPAGE® LDS Sample buffer (ThermoFisher Scientific, Waltham, Mass.).

### Western Blot

Samples were separated on 4-12% NuPAGE® gels, (ThermoFisher Scientific) using MES buffer (50 mM MES, 50 mM Tris Base, 0.1% SDS, 1 mM EDTA, pH 7.3) and transferred to nitrocellulose membrane. Membranes were blocked with 5% skim milk powder in TBS (50 mM Tris-HCl pH 7.6, 150 mM NaCl) with 0.1% Tween 20 (TBST) followed by addition of primary antibody mouse monoclonal anti-APOE (A1.4, Santa Cruz, 200 μg/ml, Dallas, Tex.) and incubated overnight at 4° C. Finally blots were incubated with horseradish peroxidase conjugated AffiniPure goat anti-mouse IgG, Fcy fragment specific (115-035-071, Jackson ImmunoResearch, West Grove, Pa.) 1 in 5000, 1 h, RT and exposed to SuperSignal® West Dura ECL reagent (ThermoFisher Scientific). Visualization was with an Amersham Imager 600 chemiluminescence imager (GE, Chicago, Ill.).

### Glycoproteomics Sample Preparation

IP samples were separated on 4-12% NuPAGE® gels (ThermoFisher Scientific) using MOPS SDS running buffer (50 mM MOPS, 50 mM Tris, 0.1% SDS, 1 mM EDTA) followed by reduction and alkylation using 50 mM DTT for 1 h at 65° C. and 125 mM Iodoacetamide 30 min, RT in the dark and the reaction stopped with 125 mM DTT. Standard plasma APOE (rPeptide) was included on gels.

All solvents were of MS quality. APOE bands were excised including gel to account for low abundance modifications. Bands were washed until destained (40 min at 37° C., 100 mM ammonium bicarbonate twice followed by 50% acetonitrile in 100 mM ammonium bicarbonate twice until destained) and dried before trypsinization with 500 ng of Trypsin Gold, MS grade trypsin (Promega, Madison, Wis.) for 16 hours, 37° C. to ensure adequate trypsinization of APOE. Peptides and glycopeptides were extracted with water followed by 50% acetonitrile/0.1% trifluoroacetic acid and samples dried ready for MS analysis.

### Mass Spectrometric Method

MS analyses were carried out on a TripleTOF© 6600 QTOF (Sciex, Concord, Ontario, Canada), used in positive ion mode. A NanoACQUITY UPLC (Waters, Milford, Mass.) with an analytical ACQUITY UPLC M-Class peptide BEH C18 column (300 Å, 1.7 μm, 75 μm×15 cm, Waters) and a nanoACQUITY UPLC symmetry C18 trap column (100 Å, 5 μm, 180 μm×20 mm, Waters) was used. Mobile phases, solvent A (aqueous 2% acetonitrile, 0.1% formic acid) and solvent B (acetonitrile, 0.1% formic acid) were used for a 60 minute gradient with a trapping flow rate of 15 μl/min and analytical flow rate of 400 nl/min. The gradient began with 1 minute at 99% solvent A and an increase of solvent B from 5 to 50% in 35 minutes increased to 99% solvent B in 2 minutes held for 3 minutes before returning to 99% solvent A for 20 minutes. Declustering potential was set to 80 and ionspray voltage 2300. A top 30 data-dependent acquisition method was used. TOF MS accumulation time of 250 ms, 400-1250 Da. The TOF MSMS accumulation time 50 ms, 100-1500 Da, intensity threshold of 100 based on background and exclusion after 2 MS/MS of 5 seconds based on peak width. The method allowing for at least 10 points on the curve of the narrowest peak under analyses. Independent data acquisition (IDA) collision energy parameters were set as follows, written as charge, slope and intercept; unknown, 0.049 and −1; 1, 0.05, 5; 2, 0.049, −1; 3, 0.048, −2; 4 and higher, 0.05, −2.

A blank was run after every sample to stop any unexpected carry over interference and standard (trypsinised β-galactosidase) was run following every fourth sample. Sample order was randomised. Standard APOE from plasma (rPeptide) was also run to ensure glycopeptide detection and expected separation.

All APOE glycopeptides in each sample were confirmed manually by parent m/z, MSMS and relative retention time. All glycopeptide spectra contained strong glycan (oxonium) ions indicating structure. A large range of glycan structures were searched for on the possible glycopeptides of APOE. Core 1 structures including the sialylated and disialylated forms as well as extended forms with and without NeuAc. Truncated core 1 structures included Tn antigen (GalNAc) and sialyl Tn. A range of core 2 structures including sialylated forms as well as extended core 2 structures with and without the addition NeuAc. Peptides with more than one possible glycosylation site were searched for as containing multiple glycans, focusing on glycans that may have been identified previously on that peptide. All hexosamine residues are assumed to be GalNac and hexose residues Gal, forming a core 1 structure. All MS/MS spectra shown herein for all glycoforms and structure accurately represent the level of annotation.

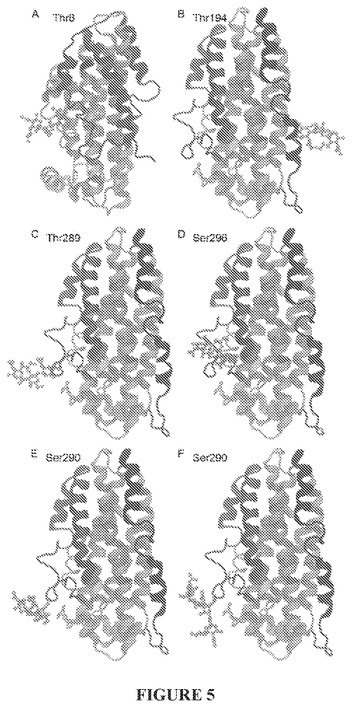

MultiQuant™ software version 2.1.1 (Sciex, Framingham, Mass.) was used for quantitation. Quantitation was based on parent mass, mass allowance was +/−0.05 Da. Two peptides, LGPLVEQGR (SEQ ID NO: 6) (amino acid 181-189, hinge region) and LQAEAFQAR (SEQ ID NO: 7) (amino acid 252-260, C-terminal) that cannot hold glycosylation were monitored for total APOE quantitation, chosen based on previous data showing them to be the most consistently intense peptides in all sample types. Data is shown as relative quantitation where area under the curve was determined for non-glycosylated and all glycoforms of each a given peptide and a total determined. The relative percentage of each peptide was then determined and is show in Table 1 for each sample and in FIG. 4B.

TABLE 1
Patient information. Plasma samples were collected in EDTA
and CSF by lumbar puncture. After collection, samples were
aliquoted to reduce freeze thaw cycles and stored at −80° C.
All patients were control individuals in Alzheimer's disease
studies and gave written informed consent.

| | CSF 1 | CSF 2 | Plasma 1 | Plasma 2 |
|---|---|---|---|---|
| Age at sampling | 57.2 | 54.8 | 68 | 67 |
| Sex | Female | Male | Female | Male |
| APOE genotype | 3.3 | 4.4 | 3.3 | 3.3 |
| PiB PET | Neg | Neg | — | — |
| MMSE | — | — | 30 | 30 |

### Glycoprotein Modelling

Glycoprotein modelling was performed using a prototype of the new GLYCAM-Web glycoprotein builder (www.glycam.org/gp) (GLYCAM-Web-glycoprotein builder (Woods Group, Complex Carbohydrate Research Center, University of Georgia, Athens, G A, 2005-2017), which uses the GLYCAMO6 forcefield to generate 3D structures of carbohydrates. The full length APOE3 NMR structure was downloaded from the Research Collaboratory for Structural Bioinformatics Protein Data Bank (PDBID: 2L7B). Each 3D glycan structure was superimposed onto the appropriate Ser/Thr sidechain of APOE3. Any atomic overlaps between the glycan and the protein were relieved by adjusting the protein sidechain and glycosidic linkage dihedral angles. The glycosylated APOE3 structures were visualised using Visual Molecular Dynamics software (VMD 1.9.3).

### N-Terminal Glycosylation

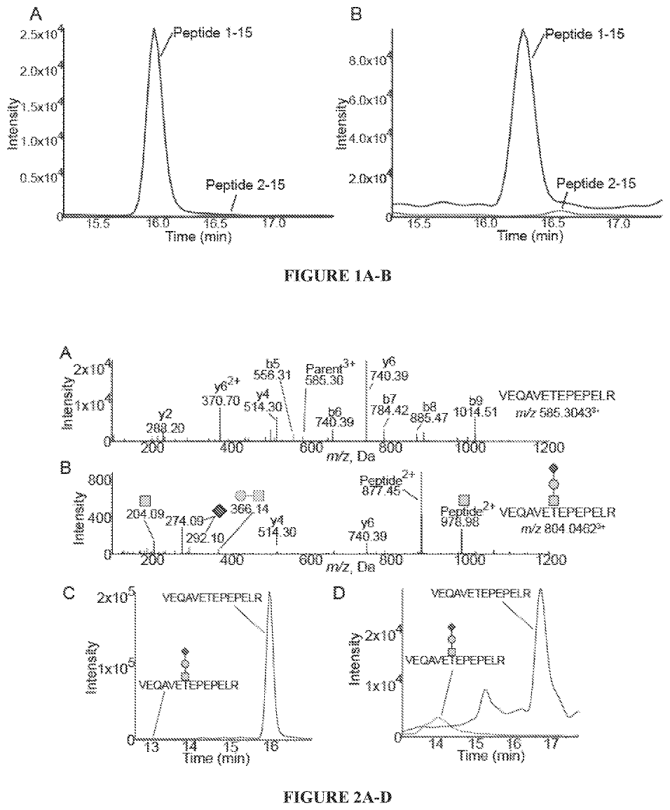

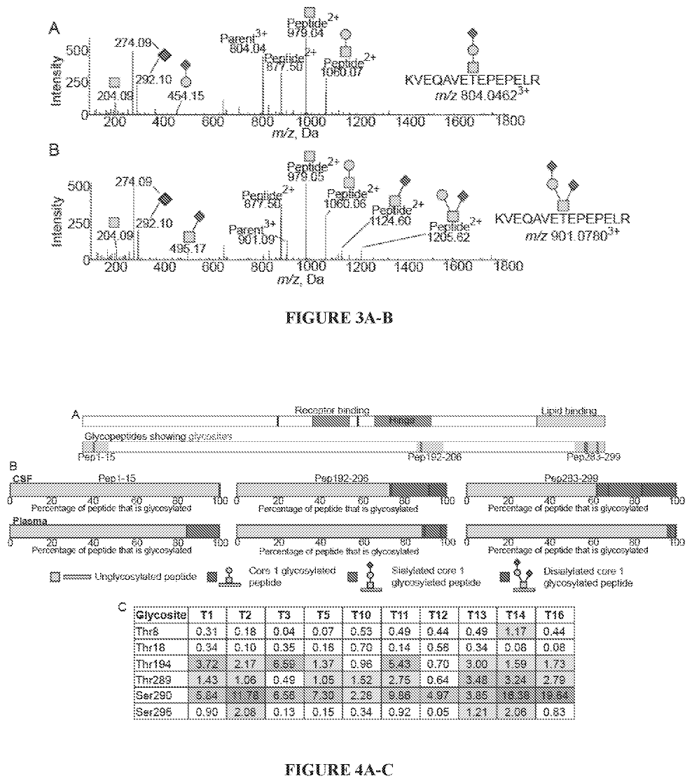

APOE was isolated by immunoprecipitation from normal adult CSF (n=2, mean age 56 yr) and plasma (n=2, mean age 67.5 yr) of known APOE genotype (Table 1). APOE tryptic peptides and glycopeptides were analyzed using a top 30 method using a TripleTOF© 6600 QTOF (Sciex) allowing at least 10 points on the curve. The N-terminal tryptic peptide 1-15 was found to be glycosylated at Thr8, the only possible site on the peptide sequence, KVEQAVETEPEPELR (SEQ ID NO: 1), FIG. 1. The first lysine residue of the mature APOE protein was not trypsinized; this was compared in each sample and the pep2-15 was found to be orders of magnitude lower in abundance compared to the pep1-15 (FIG. 1). The peptide (FIG. 2A) was identified at the expected parent mass of an attached sialylated core 1 structure. MS/MS of this parent (FIG. 2B) gave NeuAc (m/z 274.09 and 292.10) and Gal-GalNAc (m/z 366.15) fragments confirming the sialylated core 1 structure. The O-glycan attached was confirmed to be the NeuAcα2-3Galβ1-3GalNAcα1- linear form by the identification of the NeuAc-Gal peak at m/z 454.15 (FIG. 3) using an APOE plasma standard and a targeted MS method specific for these glycopeptides. No other attached glycan could be identified and confirmed by MS/MS in the samples tested, although the disialylated core 1 structure was able to be detected in the standard APOE at high concentrations using a directed method (FIG. 3).

While the sole identified glycan attached was the NeuAcα2-3Galβ1-3GalNAcα1-structure, the site occupancy was different between CSF and plasma. Quantitation by area under the curve was determined from extracted ion chromatogram (XIC) and relative quantitation shown for each form of the peptide. CSF APOE showed very low glycosylation, with the NeuAcα2-3Galβ1-3GalNAcα1- pep1-15 only 0.19% of the total quantified pep1-15 (FIG. 2C, FIG. 4B). Plasma on the other hand showed a high relative abundance of the glycosylated form: 15.80% of pep1-15 was glycosylated with the NeuAcα2-3Galβ1-3GalNAcα1- structure (FIG. 2D, FIG. 5A).

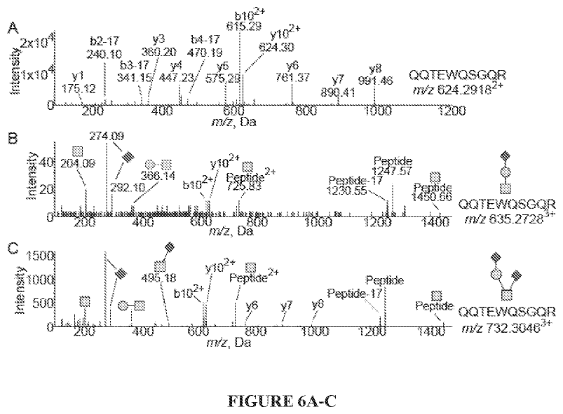

An additional low occupancy N-terminal glycopeptide, pep16-25, the next tryptic peptide, was also identified, but only when a high amount of standard APOE was analyzed. The peptide with sialylated core 1 and disialylated core 1 structures attached was identified, with the disialylated the more abundant. Fragmentation data is shown in FIG. 6. This glycosylation was not identified in the normal adult samples.

### Hinge Glycosylation

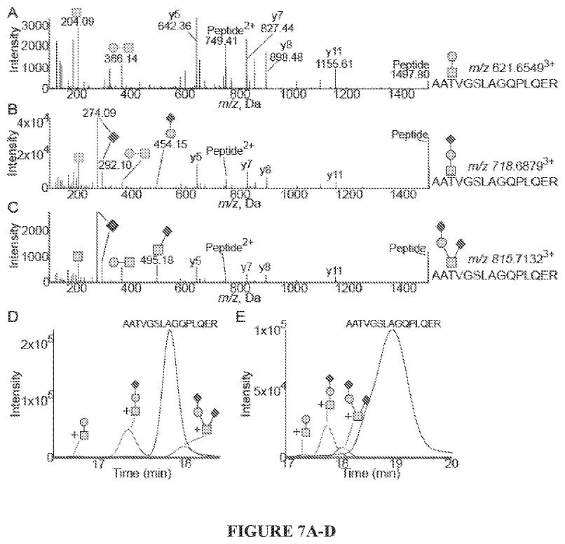

The flexible hinge region was identified to contain a single glycopeptide, pep192-206, AATVGSLAGQPLQER (SEQ ID NO: 4). Although the glycopeptide shows two possible glycosites there have been multiple studies identifying Thr194 as a glycosite of APOE. The single glycosylation site was identified to hold three glycosylation types: core 1 (FIG. 7A), sialylated core 1 (FIG. 7B), and disialylated core 1 structures (FIG. 7C). The distinction between the sialylated and disialylated core 1 glycosylated peptide is apparent by the difference in parent mass as well as relative intensity of sialic acid peaks (m/z 274.09 and 292.10) and the y-series ions. The MS/MS of the unglycosylated pep192-206 is shown in the Supplementary Fig S4. The linear NeuAcα2-3Galβ1-3GalNAcα1- structure was again confirmed by the very low abundance m/z 454.15 fragment (FIG. 7B). The NeuAcα2-3Galβ1-3(NeuAcα2-6)GalNAcα1- structure was confirmed by the presence of the NeuAcα2-3GalNAc fragment observed at m/z 495.18 (FIG. 7C).

The relative proportions of the three glycans were similar between the CSF (FIG. 7D) and plasma (FIG. 7E) APOE, with the highest proportion of NeuAcα2-3Galβ1-3GalNAcα1- glycosylated pep192-206 followed by the NeuAcα2-3Galβ1-3(NeuAcα2-6)GalNAcα1- structure and finally, a very small proportion of the Galβ1-3GalNAcα1-structure. The site occupancy, however, differs between the source of APOE (FIG. 4B): CSF APOE holds an overall 26.82% of glycosylated pep192-206 (disialylated 8.24%, sialylated 17.77%, unmodified 0.81%) compared to only 11.40% in plasma APOE (disialylated 2.62%, sialylated 7.43%, unmodified 1.35%).

### Lipid Binding Domain

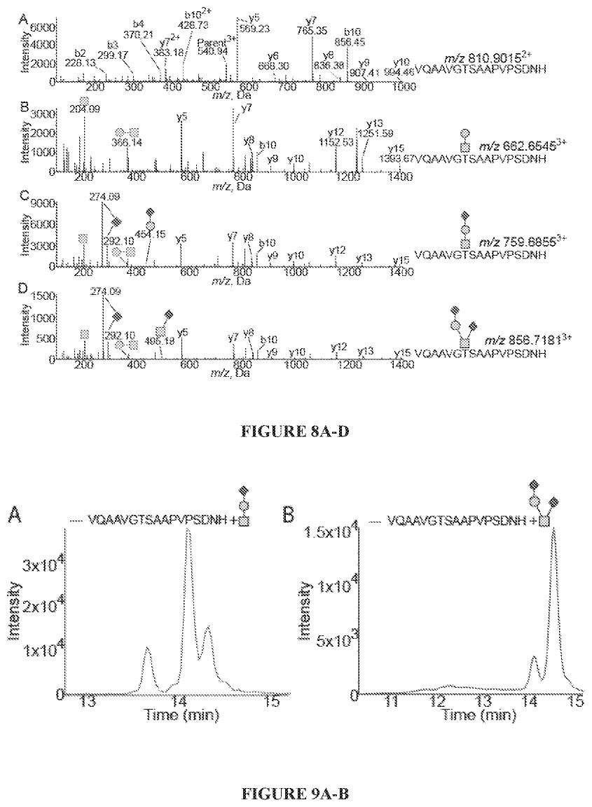

One peptide within the lipid binding domain was identified as 0-glycosylated: pep283-299, the C-terminal peptide, VQAAVGTSAAPVPSDNH (SEQ ID NO: 5). The peptide was identified to hold only core 1 structures including the unmodified, sialylated and disialylated forms (MS/MS in FIG. 8). The peptide contains three possible glycosites, Thr289, Ser290 and Ser296. All three sites were found to be glycosylated in CSF APOE (FIG. 9). However, only a single site was found to be glycosylated on any given peptide, resulting in three MS/MS-confirmed chromatographic peaks for each of the NeuAcα2-3Galβ1-3GalNAcα1- (FIG. 9A) and NeuAcα2-3Galβ1-3(NeuAcα2-6)GalNAcα1- forms (FIG. 9B). Although collision-induced fragmentation (CID), as used here, is ideal for determining both peptide and glycan structure, it is not optimal for determining attachment site, and the obtained CID MS/MS spectra were unable to differentiate the three pep283-299 isomeric peaks (FIG. 9). The glycosylation on plasma APOE was less intense and a single chromatographic peak was identified for each glycoform.

The APOE isolated from CSF held approximately ten times more total pep283-299 glycosylation (37.79%) compared to plasma pep283-299 (3.65%, FIG. 4B). The glycan proportions were also different; plasma pep283-299 had a lower proportion of the core 1 disialylated structure (disialylated 0.12%, sialylated 2.13%, unmodified 1.40%) compared to CSF pep283-299 (disialylated 16.45%, sialylated 15.62%, unmodified 5.72%).

### Comparison of Glycosylation and GalNAc-T Preference Between CSF and Plasma APOE

Relative quantitation of each glyco-variant of the analyzed glycopeptides is shown as a percentage for each of the total of all variants identified for that peptide from CSF and plasma samples (individual sample data in Table 2). APOE from the two samples, CSF and plasma, show considerable glycosylation differences (FIG. 4B). Plasma APOE has a greater abundance of glycosylation at the N-terminal region and CSF APOE has a greater abundance of glycosylation at the C-terminal region, mainly in the lipid binding domain. Both CSF and plasma APOE are glycosylated in the hinge region, although to a greater extent on CSF APOE (FIGS. 4A and 4B).

TABLE 2
Relative quantitative data for individual CSF and plasma samples.
All data are shown as percentages.

| Peptide | CSF 1 | CSF 2 | Plasma 1 | Plasma 2 |
|---|---|---|---|---|
| 1-15 pep | 99.90 | 99.90 | 86.97 | 81.43 |
| 1-15 sialyl core 1 | 0.10 | 0.10 | 13.03 | 18.57 |
| 192-206 pep | 71.55 | 74.81 | 90.55 | 86.63 |
| 192-206 core 1 | 0.69 | 0.94 | 1.35 | 1.35 |
| 192-206 sialyl core 1 | 18.90 | 16.63 | 5.81 | 9.05 |
| 192-206 disialyl core 1 | 8.86 | 7.62 | 2.29 | 2.96 |
| 283-299 pep | 60.51 | 63.89 | 95.58 | 97.12 |
| 283-299 core 1 | 7.09 | 4.36 | 1.81 | 1.00 |
| 283-299 sialyl core 1 | 15.01 | 16.23 | 2.44 | 1.82 |
| 283-299 disialyl core 1 | 17.39 | 15.52 | 0.18 | 0.06 |

The suitability of each glycosite to be a substrate for specific GalNAc-Ts, GalNAc-T1-3, 5, 10-14 and 16, was also analyzed using ISOGlyP. ISOGlyP site preference results (FIG. 4C) are given as EVP (enhancement value product): the likelihood the given GalNAc-T contributed to glycosylating that glycosite, where below 1 the GalNac-T is unlikely to have contributed and above 1 the likelihood is enhanced. The N-terminal sites Thr8 and Thr18 were all below 1 for the available enzymes except for Thr8 and GalNAc-T14, which was slightly positive with an EVP 1.17. Thr-194 was shown to prefer the ubiquitous GalNAc-T1 (EVP 3.72) as well as T3 (EVP 6.59) and T11 (EVP 5.43). The C-terminal glycosites instead had a preference for less common GalNAc-Ts. Thr289 showed a preference for T13, T14 and T16 (EVP 3.48, 3.24, 2.79) and Thr290 showed a marked preference for T16 (EVP 19.64), followed by T14 (EVP 16.38) and T2 (EVP 11.78). Thr296 showed less GalNAc-T specificity overall though favored T2, T14 and T13 (EVP 2.08, 2.06, 1.21). These data suggest that the common hinge glycosite Thr194 and the CSF dominant C-terminal glycosites (Thr289, Ser290 and Ser296) are preferential substrates for a different subset of GalNAc-Ts.

### Modeling of CSF and Plasma Glyco-APOE

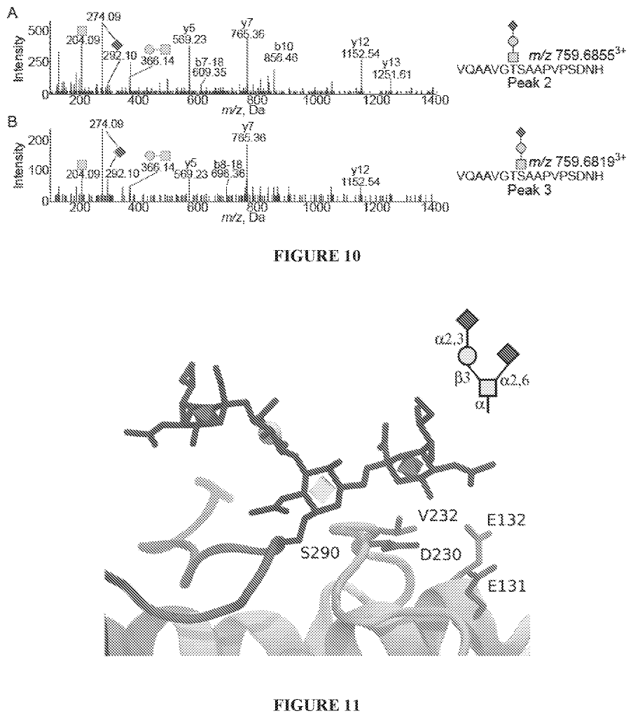

To understand how the characterized glycans interrelate with the protein backbone, GLYCAM-Web glycoprotein builder was used to model identified glyco-APOE structures. The full-length APOE3 NMR structure (PDBID: 2L7B) was used for all structures. Models were made of the most abundant biologically relevant CSF and plasma APOE glycoforms (FIG. 5). N-terminal Thr8 is a buried residue situated at the posterior side of helix N1 (AA 6-9) making it restrictive to the GalNAc of the glycan structure at this site (FIG. 5A). The linear glycan extends out from the space between helix N1 and helix 1 (AA 26-40); a more detailed view of the glycosylated Thr8 is shown in FIG. 10. The hinge region Thr194 residue is widely open and can hold the core 1, sialylated and disialylated structures. The sialylated core 1 Thr194 glycoform, the most abundant Thr194 glycoform in both the CSF and plasma, is shown in FIG. 5B.

The lipid binding domain pep283-299 holds glycosites Thr289, Ser290, and Ser296; each glycosite is individually glycosylated on a given peptide in normal, adult CSF. The three C-terminal glycosites with the same NeuAcα2-3Galβ1-3GalNAcα1- structure attached were modeled and it was found that the angle at which the attached glycan extends differs between the glycosites, even between adjacent Thr289 (FIG. 5C) and Ser290 (FIG. 5E) residues. These differences are due to the glycosite positions on the rigid curve of loop C which does not move with the addition of the flexible glycan structure. This rigidity is also true for the Ser296 (FIG. 5D) glycosite which is positioned very close to the C-terminal (AA 299); no backbone change is observed with the attachment of the sialylated or disialylated core 1 structures. These three structures show the linear NeuAcα2-3Galβ1-3GalNAcα1- structure. These structures hold a negatively charged sialic acid at the terminus of the large glycan structure, extended from the protein backbone to which the glycan structure is attached. The addition of the α2-6 NeuAc residue to the GalNAc, forming the NeuAcα2-3Galβ1-3(NeuAcα2-6)GalNAcα1- structure, dramatically changes the shape of the once linear, now branched glycan structure (compare FIGS. 5E and 5F). This extension also positions a negatively charged NeuAc residue closer to the protein backbone, as shown in more detail in FIG. 11, the NeuAcα2-3Galβ1-3(NeuAcα2-6)GalNAcα1- glycosylated Ser290 glycosite. The α2-6 NeuAc residue has the potential to interact with the protein backbone observed here by its proximity not only to the adjacent amino acid side chains, but also other amino acids of the C-terminal domain including to side chains from other C-terminal domain amino acids outside the lipid binding domain, V232 and D230, and from the N-terminal domain, E132 and E131.

The studies described herein showed that APOE glycosylation varies dramatically between the CSF and plasma. CSF APOE is more heavily glycosylated and holds substantially more sialylated and disialylated core 1 structures within the C-terminal lipid binding domain. In contrast, plasma APOE holds the sialylated core 1 O-glycan on the N-terminal domain. Both APOE from the CNS and the periphery hold glycosylation at the hinge Thr194 site with similar glycan distributions, though the CSF APOE to a greater extent. This APOE glycosylation dichotomy has important implications, not only on the mechanism of CNS and systemic APOE tailoring lipoprotein binding specificity but also has the potential to extend into its critical role in AD. This outlines our ability to monitor glyco-APOE specific changes and the novel possibility of identifying brain specific glycoforms of APOE in the plasma.

Described herein is a comprehensive analysis of all detectable identified glycosites of normally glycosylated APOE from the plasma and CSF, including the attached glycans and site occupancy, allowing a greater fundamental understanding of the APOE molecule around the body. This was achieved using 25 μl of CSF. The site occupancy for most sites may appear relatively low; however, two things should be taken into consideration. First, it has been suggested that APOE is more heavily glycosylated when in the cell compared to secreted forms of the protein, which would include both the CSF and plasma samples studied here. Second, relative quantitation is hampered by the suppression of sialic acid holding peptides in positive ion mode. This suppression is reduced with the use of nano-ESI as used herein.

The hinge glycosylation of pep192-206, glycosite Thr194, is most similar between the CSF and plasma samples. The hinge flexibility is essential for unfolding of APOE and, as the glycosite is situated on the Hinge H2 helix, rather than on the intervening loop regions, it would not likely affect unfolding. Thr8 N-terminal glycosylation, however, was more abundant on plasma APOE compared to CSF APOE and the Thr18 glycosite was below detection in the samples tested (though identified in a similarly processed APOE standard). The Thr18 site has previously required unique sample preparation conditions for identification. Both glycosites are buried: Thr8 is located at the interior side of the N1 helix and Thr18 at the interior side of the second turn of the N2 Helix. Accessibility of the more abundant Thr8 glycosite is not improved by the first step of APOE unfolding, which is fast and reversible. It is exposed by the second step of unfolding; however, this is slow and, although reversible, requires lipid or heparin binding, making it unlikely to occur for the addition of O-glycosylation. The close proximity of Thr8 to the N-terminal suggests that there may be inherent flexibility allowing GalNAc-T accessibility to the glycosite.

The C-terminal glycosylation is situated within the lipid binding region and thus is of great import for APOE function. It also shows the greatest difference between the two sample types, with CSF APOE having much greater glycosylation in the lipid binding region. The lipoprotein binding profiles of plasma APOE and CSF APOE also differ markedly, as plasma APOE must bind to lipoproteins with large ranges of size, shape and composition, while APOE in the CNS interacts only with HDL. HDL from the brain is elliptical and small (8-15 nm) while in the CSF it is larger (12-20 nm, with a small population up to 30 nm) and spherical. APOE in the plasma, however, has a much larger range of binding particles, including the very large chylomicrons (75-1200 nm) which reduce in size to remnant particles (30-80 nm). Plasma APOE also binds large polyhedral VLDL (30-100 nm), which also change in size and shape to the IDL (25-35 nm) stage and small plasma HDL particles (7-14 nm). In order for one protein to be able to bind all of these structures, even as they change in size, is exceptionally accommodating, the reduced C-terminal glycosylation of plasma APOE gives a less encumbered lipid binding domain.

The removal of amino acid 244 onwards (from middle of Helix C2) removes all lipoprotein binding of APOE, however, the binding preferences of this region are lipoprotein specific. The C-terminal region (AA 273-299), made up of the helix C3 (AA 271-276) and loop C (AA 277-299), has been shown to mediate the binding to HDL. This binding preference is APOE genotype dependent, with the APOE4 protein more dependent on the C-terminal 273-299 region than APOE3. The upstream portion of the lipid-binding region is more necessary for VLDL and LDL binding. Self-association is also completely terminated by the loss of the AA273-299 region for APOE4 but not for APOE3. Self-association may be important for the construction of large complexes with HDL particles able to hold at least two APOE proteins; APOE4 positive genotypes produce smaller lipoprotein particles in normal, adult CSF compared to APOE4 negative genotypes. Therefore changes in the C-terminal region 273-299 may affect HDL binding more dramatically than other lipoproteins, as well as self-association, and affect the APOE4 protein to a higher degree than the APOE3 protein.

APOE glycosylation and, in particular, sialylation, affects the lipoprotein binding preference of APOE. In a study of APOE glycosylation on lipoprotein binding, APOE was de-sialylated with a neuraminidase that preferentially removes α2-3 linked NeuAc but also α2-6 linked NeuAc (as shown herein, the most common sialic acid residues on APOE). A comparison in the binding of sialylated and de-sialylated APOE revealed that HDL bound five times more effectively to sialylated APOE, while VLDL bound only two times more effectively to sialylated APOE (Marmillot et al., Metabolism: Clinical and Experimental 48: 1184-1192 (1999)). HDL binding strength of de-sialylated APOE was rescued by the re-addition of sialic acid using sialyltransferases from rat liver golgi, confirming the significance of glycosylation with the charged NeuAc to HDL binding. De-sialylation would remove NeuAc from all APOE glycosites, thus indicating the importance of the N-terminal or hinge glycosites. However, the low abundance of N-terminal glycosylation in the CSF, compared to the high abundance in the plasma APOE, indicates it may not be essential for HDL binding. Also, the glycosites within the lipid-binding region are the most NeuAc-rich, the only APOE region showing equivalent amounts of the NeuAcα2-3Galβ1-3GalNAcα1- and NeuAcα2-3Galβ1-3(NeuAcα2-6)GalNAcα1- structures, while all others show a marked preference for the NeuAcα2-3Galβ1-3GalNAcα1- structure. This is particularly relevant as sialyltransferases decrease with age and in AD, suggesting an explanation for APOE HDL binding deficiencies over time and in AD.

The addition of O-glycosylation can have chemical effects on the protein backbone, and the location of glycosylation can have wide reaching changes on the protein structure. However, based on the modeling studies described herein, C-terminal APOE glycosylation does not alter the protein backbone, instead, the glycan contours around the protein backbone. It is also possible that the addition of the backbone adjacent 2-6 linked NeuAc could interact with the protein backbone, and there is potential to interact across APOE domains and alter the unfolding properties of APOE.

In summary, the C-terminal loop structure is an important domain for tailoring lipoprotein binding preference and this domain remains almost exclusively unglycosylated within the plasma, leaving it open for the wide range of lipoproteins that bind there. In the CNS, however, although the loop C protein backbone structure remains unchanging, the higher abundance glycosylation appears to tailor HDL binding by the addition of negatively charged sialylated glycans previously shown to improve HDL binding. This fundamental observation has widespread implications given the critical importance of APOE in Alzheimer's disease. The low abundance C-terminally glycosylated plasma APOE is a reflection of brain APOE state, and thus presents a way of systematically sampling differences in brain APOE across individuals, with aging, or after interventions that may alter APOE. Given that there are factors that may affect glycosylation in aging through the reduction in sialyltransferases over time, as well as in AD, monitoring systemically available glyco-APOE provides opportunities to evaluate changes in brain APOE in a clinically applicable method.

Treatment with Anti-Inflammatory Agents

APOE3 and APOE4 knock-in mice were examined for evidence of impairment of brain function in the absence of overt damage from Alzheimer's disease or other age-related disorders. APOE4 mice showed reduced neuronal complexity (as measured by Golgi staining) and delayed ability to achieve a spatial learning task, the Barnes maze. The APOE protein in APOE4 mouse brain showed a different pattern of isolation compared to APOE3 mouse brain, with more APOE readily soluble from APOE4 brains; similar findings were made in samples of human brains. It was found that readily soluble APOE was characterized by post-translational modifications as determined by one-dimensional and two-dimensional gel electrophoresis. As set forth above, glycosites and the attached glycans of the APOE protein, using samples of human cerebrospinal fluid and plasma, were identified. Using a novel mass spectrometry method, glycosylation of APOE Thr-194, as well as substantial modification of APOE at other sites, were found. APOE4 animals were treated with 375 ppm ibuprofen in chow for two months, and its effects on behavior and neuron structure were measured. Ibuprofen-treated APOE4 mice showed significantly higher levels of neuron complexity, as measured by dendritic spine density, and significantly improved spatial learning, as measured by the Barnes maze. Similar effects on neuronal complexity were found with a one-week treatment of APOE4 mice. These data suggest that the contribution of APOE genotype to Alzheimer's disease risk is related to an effect on predisposition of the brain to inflammation.

What is claimed is:

1. A method of detecting a cerebrospinal fluid-specific (CSF-specific) glycoform of Apolipoprotein E (APOE) in a subject comprising:
a) obtaining a plasma sample from the subject; and
b) detecting a CSF-specific glycoform of APOE in the plasma sample.

2. The method of claim 1, further comprising determining the level of the CSF-specific glycoform of APOE and/or the glycosylation pattern of the CSF-specific glycoform of APOE in the plasma sample.

3. The method of claim 1, wherein the subject has at least one copy of the APOE4 allele.

4. The method of claim 1, wherein the subject lacks copies of the APOE4 allele.

5. The method of claim 1, wherein the CSF-specific glycoform of APOE is an APOE glycoform that differs in glycosylation as compared to a control plasma-specific glycoform of APOE.

6. The method of claim 1, wherein the CSF-specific glycoform of APOE is an APOE glycoform that differs in glycosylation as compared to a control CSF-specific glycoform of APOE.

7. The method of claim 5, wherein the difference in glycosylation is a difference in the glycosylation pattern of the CSF-specific APOE glycoform.

8. The method of claim 7, wherein the difference in the glycosylation pattern is a difference in the number of glycosylated O-linked glycosylation sites, a difference in the type of O-glycan at one or more glycosylation sites, a difference in the amount of glycosylation at one or more O-linked glycosylation sites and/or a difference in sialylation at one or more O-linked glycosylation sites.

9. The method of claim 8, wherein the difference in the glycosylation pattern is a difference in the amount of glycosylation, type of O-glycan, and/or amount of sialyation at Thr8, Thr18, Thr194, Ser197, Thr289, Ser290 and/or Ser296 of the CSF-specific APOE glycoform.

10. The method of claim 9, wherein the difference in the glycosylation pattern is a difference in the amount of glycosylation, type of glycan, and/or amount of sialyation at Thr289, Ser290 and/or Ser296 of the CSF-specific APOE glycoform.

11. The method of claim 1, further comprising detecting a plasma-specific glycoform of APOE.

12. (canceled)

13. The method of claim 1, wherein a plasma-specific form of ApoE is not detected.

14. (canceled)

15. The method of claim 1, wherein two or more CSF-specific APOE glycoforms are detected.

16. A method of diagnosing Alzheimer's disease or a risk of Alzheimer's disease in a subject comprising:
a) obtaining a plasma sample from a subject;
b) detecting a CSF-specific glycoform form of APOE in the plasma sample;
c) diagnosing the subject as having Alzheimer's disease or at risk of developing Alzheimer's disease when a difference in the level of the CSF-specific form of APOE, and/or the glycosylation pattern of the CSF-specific form of APOE as compared to a control level of the CSF-specific form of APOE, and/or a control glycosylation pattern of the CSF-specific form of APOE is detected.

17. (canceled)

18. (canceled)

19. The method of claim 16, wherein an increase or a decrease in the level of the CSF-specific form of APOE is detected.

20. (canceled)

21. (canceled)

22. The method of claim 16, wherein the difference in the glycosylation pattern is a difference in the amount of glycosylation, type of glycan, and/or amount of sialyation at Thr8, Thr18, Thr194, Ser197, Thr289, Ser290 and/or Ser296 of the CSF-specific APOE glycoform.

23. The method of claim 22, wherein the difference in the glycosylation pattern is a difference in the amount of glycosylation, type of glycan, and/or amount of sialyation at Thr289, Ser290 and/or Ser296 of the CSF-specific APOE glycoform.

24. (canceled)

25. The method of claim 16, wherein an increase in the amount of glycosylation at one or more O-linked glycosylation sites of the CSF-specific form of APOE is detected.

26. The method of claim 16, wherein an increase in the amount of glycosylation at one or more O-linked glycosylation sites and a decrease in the amount of glycosylation at one or more O-linked glycosylation sites is detected.

27. The method of claim 16, wherein an increase or a decrease in the amount of sialylation at one or more O-linked glycosylation sites of the CSF-specific form of APOE is detected.

28. (canceled)

29. The method of claim 22, wherein an increase in the amount of sialylation at one or more O-linked glycosylation sites and a decrease in the amount of sialylation at one or more O-linked glycosylation sites is detected.

30. (canceled)

31. (canceled)

32. The method of claim 16, further comprising administering one or more agents that slows the progression or delays the development of Alzheimer's disease.

33. The method of claim 32, wherein the one or more agents are selected from the group consisting of a nonsteroidal anti-inflammatory drug (NSAID), a tyrosine kinase inhibitor, an acetylcholinesterase inhibitor, and an NMDA receptor inhibitor.

34. A method of determining the progression of Alzheimer's disease or an increase in the risk of developing Alzheimer's disease in a subject comprising:
a) obtaining a first plasma sample from the subject;
b) detecting a level and/or a glycosylation pattern of a CSF-specific glycoform of APOE in the plasma sample;
c) obtaining a second plasma sample from the subject;
d) detecting a second level and/or second glycosylation pattern of a CSF-specific form of APOE in the plasma sample;
e) comparing the first level and/or glycosylation pattern of the CSF-specific glycoform of APOE with the second level and/or glycosylation pattern of the CSF-specific glycoform of APOE, wherein if the level and/or glycosylation pattern of the CSF-specific glycoform of APOE in the second biological sample is more similar to control indicating the progression of Alzheimer's disease or increased risk of Alzheimer's disease, as compared to the first biological sample, Alzheimer's disease has progressed or the risk of developing Alzheimer's disease has increased in the subject.

35. (canceled)

36. The method of claim 34, further comprising administering one or more agents that slows the progression or delays the development of Alzheimer's disease.

37. (canceled)

38. A method for determining the efficacy of a selected treatment for slowing the progression or delaying the development of Alzheimer's disease in a subject comprising:
a) obtaining a plasma sample from the subject before the selected treatment;
b) detecting a level and/or a glycosylation pattern of a CSF-specific glycoform in the sample of step (a);
c) treating the subject with the selected treatment;
d) obtaining a plasma sample from the subject after the selected treatment;
e) detecting a level and/or glycosylation pattern of a CSF-specific glycoform of APOE in the sample from step (d);
f) comparing the level and/or glycosylation pattern of the CSF-specific glycoform detected in step (b) and (e) to determine whether the level and/or glycosylation pattern is the same or whether the level and/or glycosylation pattern detected in step (b) or (e) is more similar to control, a level and/or glycosylation pattern in step (e) more similar to control indicating that the selected treatment is effective for treating or preventing Alzheimer's disease.

39. The method of claim 38, wherein the CSF-specific form of APOE is detected by an immunoassay or mass spectrometry.

40. (canceled)


SEQUENCE LISTING (7 sequences)

SEQ ID NO: 1 - protein, 15 aa, Artificial sequence
  Synthetic construct
KVEQAVETEPEPELR

SEQ ID NO: 2 - protein, 14 aa, Artificial sequence
  Synthetic construct
VEQAVETEPEPELR

SEQ ID NO: 3 - protein, 10 aa, Artificial sequence
  Synthetic construct
QQTEWQSGQR

SEQ ID NO: 4 - protein, 15 aa, Artificial sequence
  Synthetic construct
AATVGSLAGQPLQER

SEQ ID NO: 5 - protein, 17 aa, Artificial sequence
  Synthetic construct
VQAAVGTSAAPVPSDNH

SEQ ID NO: 6 - protein, 9 aa, Artificial sequence
  Synthetic construct
LGPLVEQGR

SEQ ID NO: 7 - protein, 9 aa, Artificial sequence
  Synthetic construct
LQAEAFQAR